(19) United States
(12) Patent Application Publication
(10) Pub. No.: US 2025/0057402 A1
(43) Pub. Date: Feb. 20, 2025

(54) CAPSULE ENDOSCOPE

(71) Applicants: Ankon Medical Technologies(Shanghai) Co., LTD, Shanghai (CN); ANX IP HOLDING PTE. LTD., SG (SG)

(72) Inventors: Xiaodong DUAN, Plano, TX (US); Junjie WANG, Shanghai (CN)

(73) Assignees: Ankon Medical Technologies(Shanghai) Co., LTD, Shanghai, CN (CN); ANX IP HOLDING PTE. LTD., SG, SG (SG)

(21) Appl. No.: 18/726,113

(22) PCT Filed: Dec. 29, 2022
(86) PCT No.: PCT/CN2022/143651

§ 371 Date: Jul. 1, 2024

(30) Foreign Application Priority Data
Dec. 31, 2021 (CN) 202111667473.3

(51) Int. Cl. A61B 1/04; A61B 1/00; A61B 1/045; A61B 1/06

(52) U.S. Cl. CPC A61B 1/041; A61B 1/00032; A61B 1/045; A61B 1/06

(57) ABSTRACT

The present invention provides a capsule endoscope, including: a power supply component; a power utilization component, comprising an image acquisition component; at least one power storage component, which is connected in parallel with the power supply component to form a power pack, the power storage component and the power supply component jointly providing power for the image acquisition component. According to the capsule endoscope, a capacitor, the power supply component and the like work in conjunction to supply power to a power utilization component, so that a peak power supply quantity can be increased, and the power utilization requirement can be met even if a high-power-consumption power utilization component operates. A multi-capacitor parallel connection mode is used in more than one power pack, so that the overlarge size of a single capacitor and possible safety risks can be avoided, and power distribution can be achieved.

### FIELD OF INVENTION

The present invention relates to the technical field of the medical device, and more particularly to a capsule endoscope.

### BACKGROUND

Capsule endoscopy is mainly used for diagnosing diseases of organs such as the stomach and intestines of the human body. The examination time in the human body can be up to ten hours, so whether the battery in the capsule endoscope can support its operation has become an important issue in this field.

At present, capsule endoscopes mainly use light emitting diode (abbreviated as LED) light sources as the illumination components for taking photos. Compared to other power-consuming components in capsule endoscopes, LED light sources consume more power. Therefore, when taking photos, the LED light source turns on, causing a significant increase in peak power consumption. If the instantaneous battery voltage is less than 1.9V, the battery may not be able to continue supplying power.

The technology threshold for capsule endoscopy is relatively high, and currently only a few companies are researching and producing it. Therefore, there are fewer solutions proposed for the power supply of capsule endoscopes. Some companies have attempted to use lithium batteries to replace traditional silver oxide batteries to provide more power and longer supply time for capsule endoscopes. However, compared to silver oxide batteries, lithium batteries have higher costs, less stable discharge, and poorer stability. Therefore, how to increase the power supply of the capsule endoscope under the premise of reducing costs and improving stability, ensuring the normal operation of the capsule endoscope throughout the entire digestive tract examination process has become a technical issue that urgently needs to be solved in this field.

### SUMMARY OF THE INVENTION

In order to solve the existing problems in the prior art, the present invention provides a capsule endoscope, comprising a power supply component and a power utilization component. The power supply component is used to supply power to the power utilization component, and the power utilization component comprises an image acquisition component. The capsule endoscope further comprises: at least one power storage component, and a first electronic switch;

the power storage component is connected in parallel with the power supply component to form a power pack, jointly providing power to the image acquisition component, where the power storage component is a single capacitor or two or more capacitors connected in parallel; and the first electronic switch is disposed between the power pack and the image acquisition component, for controlling the connection and disconnection between the power pack and the image acquisition component.

As a preferred structural form, the capsule endoscope comprises a first power storage component and a second power storage component. The image acquisition component comprises a camera component and an illumination component. The first power storage component is parallel with the power supply component to form a first power pack, supplying power to the illumination component. The second power storage component is parallel with the power supply component to form a second power pack, supplying power to the camera component.

Further, the number of capacitors in the first power pack and the second power pack is determined by the power consumption of the power utilization component they supply power to. The number of capacitors in the power pack corresponding to the high-power-consumption power utilization component is greater than the number of capacitors in the power pack corresponding to the low-power-consumption power utilization component.

Further, the first electronic switch comprises a first branch switch and a second branch switch. The first branch switch is disposed between the first power pack and the illumination component, for controlling the connection and disconnection between the first power pack and the illumination component. The second branch switch is disposed between the second power pack and the camera component, for controlling the connection and disconnection between the second power pack and the camera component.

Further, the capsule endoscope further comprises a power switch, which is disposed at the output end of the power supply component, for controlling the connection and disconnection between the power supply component and the power storage component, and the power utilization component. When the power switch is disconnected, the power supply component and the power storage component, and the power supply component and the power utilization component are both in a disconnected state.

Further, the capsule endoscope comprises a voltage regulator, which is disposed between the power pack and the image acquisition component.

Further, the voltage regulator comprises a step-up circuit and a step-down circuit, wherein the step-up circuit is disposed between the first power pack and the illumination component, and the step-down circuit is disposed between the second power pack and the camera component.

Further, the capsule endoscope comprises a second electronic switch, the power utilization component further comprises at least one functional component, the second electronic switch is disposed between the power supply component and the functional component, for controlling the connection and disconnection between the power supply component and the functional component.

Further, the capsule endoscope comprises a third electronic switch, which is disposed between the power supply component and the power storage component, the third electronic switch is used for controlling the connection and disconnection between the power supply component and the power storage component, and between the power supply component and the image acquisition component.

Further, the capsule endoscope further comprises a thermal insulation material layer, the thermal insulation material layer is disposed between the power supply component and an enclosure of the capsule endoscope.

Further, the thermal insulation material layer comprises insulating glue, aerogel material, or solid insulating material.

Further, the capsule endoscope further comprises a sealing layer, the sealing layer is disposed between the power supply component and the thermal insulation material layer.

Based on the above technical solution, the capsule endoscope disclosed by the present invention uses a combination of the capacitor, the power supply, and the voltage regulator to supply power to the power utilization components, which can increase the peak power supply capacity, thus meeting the power requirements even when high-power-consumption power utilization components are operating. A multi-capacitor parallel connection mode is used in more than one power pack, so that not only can avoid the overlarge size of a single capacitor and possible safety risks, but also allow for power distribution as needed, thereby improving the utilization rate of electrical energy.

The other features and advantages of the present invention shall be explained in detail in the subsequent detailed description of embodiments.

### BRIEF DESCRIPTION OF THE DRAWINGS

The accompanying drawings, which are included to provide a further understanding of the present invention and are incorporated in and constitute a part of the present invention, illustrate the embodiment(s) of the present invention and together with the description serve to explain the principle of the present invention. In the drawings:

FIG. 1 is a schematic diagram of a capsule endoscope according to a first embodiment of the present invention.

FIG. 2 shows a preferred implementation of the first embodiment of the capsule endoscope of the present invention.

FIG. 3 shows another preferred implementation of the first embodiment of the capsule endoscope of the present invention.

FIG. 4 is a schematic diagram of the capsule endoscope according to a second embodiment of the present invention.

FIG. 5 shows a preferred implementation of the second embodiment of the capsule endoscope of the present invention.

FIG. 6 shows another preferred implementation of the second embodiment of the capsule endoscope of the present invention.

FIG. 7 is a schematic view of the structure of the capsule endoscope according to the embodiments of the present invention.

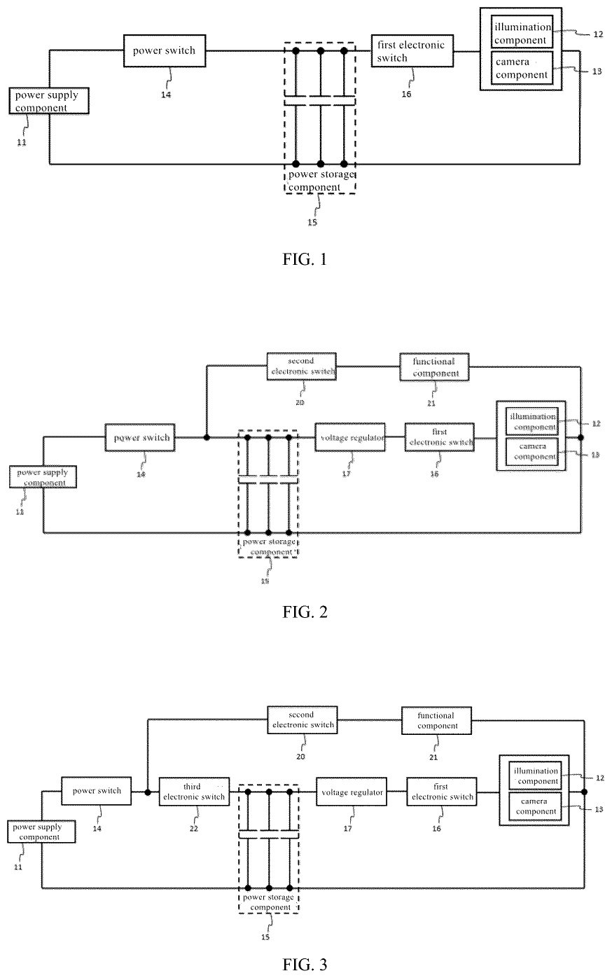

The correspondence between the marks in FIG. 1 to FIG. 7 and the names of the components is as follows:

11 power supply component; 12 illumination component; 13 camera component; 14 power switch; 15 power storage component; 16 first electronic switch; 17 voltage regulator; 18 first power storage component; 19 second power storage component; 20 second electronic switch; 21 functional component; 22 third electronic switch; 31 thermal insulation material layer; 32 sealing layer; 101 enclosure main body portion; 102, 103 enclosure end portions; 161 first branch switch; 162 second branch switch; 171 step-up circuit; 172 step-down circuit.

### DETAILED DESCRIPTION

To more clearly understand the objects, features, and advantages of the present invention, a detailed description is provided below in conjunction with the accompanying drawings and specific embodiments. It should be noted that, where there is no conflict, the embodiments and features described in the present invention can be combined with each other.

Many specific details are set forth in the following description to provide a thorough understanding of the present invention. However, the present invention can be practiced in other ways than those specifically described herein. Therefore, the scope of the present invention is not limited by the specific embodiments disclosed below.

As shown in FIG. 1, a schematic diagram of a capsule endoscope according to a first embodiment of the present invention. In this embodiment, the capsule endoscope comprises a power supply component 11, a power utilization component, and a power storage component 15. The power supply component 11 is used to supply power to the power utilization component. The power utilization component comprises or consists of an image acquisition component, the image acquisition component comprises or consists of an illumination component 12 and a camera component 13. The illumination component 12 is used to illuminate the object, which is mainly the internal tissues and organs of the human body. The camera component 13 is used to capture images inside the subject, and can be a camera. The illumination component 12 can be a lighting source suitable for illuminating, such as an LED, an organic light emitting diode (abbreviated as OLED), or any other suitable lighting source. The power storage component 15 consists of three capacitors in parallel, which are connected in parallel with the power supply component 11 to form a power pack, jointly supplying power for the illumination component 12 and the camera component 13.

In the absence of the power storage component 15, the power supply component 11 in the capsule endoscope can generally meet the long-term power supply for low-power-consumption power utilization components. However, for high-power-consumption power utilization components such as the illumination component 12 and the camera component 13, etc., a higher instantaneous energy supply is required during operation. For example, when the capsule endoscope is capturing images, the illumination component (such as LED lights) turns on, increasing the peak power consumption. If the instantaneous battery voltage is too low, generally when the instantaneous voltage is less than 1.9V, the battery may not be able to continue supplying power. Therefore, by setting the capacitors in parallel with the power supply component 11, when the illumination component 12, the camera component 13 and other high-power-consumption power utilization components are operating, using the energy storage performance of the capacitors to power the above high-power-consumption power utilization components can provide sufficient peak power, thereby ensuring the normal operation of the illumination component 12 and other high-power-consumption components.

The first electronic switch 16 is disposed between the power pack and the illumination component 12, the camera component 13 and other image acquisition components, for controlling the connection and disconnection between the power pack and the illumination component 12, and the camera component 13. When the illumination component 12 and the camera component 13 are not in operation, the first electronic switch 16 is in a disconnected state. At this time, the power supply component 11 charges the power storage component 15, but does not supply power to the illumination component 12 and the camera component 13. When the illumination component 12 and the camera component 13 are in operation, the first electronic switch 16 turns to a closed state according to the instructions of the capsule endoscope control component. The power storage component 15 starts to discharge, and at this time, the power pack composed of the power supply component 11 and the power storage component 15 jointly supplies power to the illumination component 12 and the camera component 13. By setting the first electronic switch 16, it is possible to accurately control the supply time of the peak current. Only during the operation of the illumination component 12 and the camera component 13, the power storage component 15 can be combined with the power supply component 11 to form a power pack to jointly supply power to the illumination component 12 and the camera component 13, and the circuit is disconnected when the illumination component 12 and the camera component 13 are not in operation, thereby increasing the efficiency of energy utilization under the premise of increasing the peak current and avoiding electrical energy waste.

In this embodiment, three capacitors are connected in parallel, but it should be understood that the specific number of capacitors selected is determined by the power consumption of the corresponding power utilization component, and is not limited to the above situation.

When the capsule endoscope is capturing images, the relationship between the quantity of charge and the voltage and capacitance of the power supply component 11 is as follows:

$$Q = C \times Δ U = I \times t$$

Where Q is the quantity of charge, C is the capacitance value, ΔU is the voltage difference when power supplied, I is the current required by the circuit, and t is the working time of the illumination component 12; the working time of the illumination component 12 is determined according to the actual photography needs, and the quantity of charge Q can be calculated after obtaining the time t; since the capacitance C is a fixed value, the voltage difference ΔU can be further calculated.

To increase the power supply capacity, it is possible to increase the capacitance value of capacitor C, and the goal of increasing the capacitance value can be achieved by connecting multiple capacitors in parallel. In addition, at the current stage, the size of the 220 μF capacitor has been reduced from the size of centimeters to millimeters, which provides the possibility of using large capacitors in parallel form for internal power supply of capsule endoscopes.

To illustrate by taking the replacement of three parallel 33 μF capacitors with three parallel 220 μF capacitors as an example, results in:

$$Q = C 1 \times Δ U 1 = 99 μ F \times Δ U 1$$

$$Q = C 2 \times Δ U 2 = 660 μ F \times Δ U 2$$

$$Q = 660 μ F \times Δ U 2 = 99 μ F \times Δ U 1$$

$$Δ U 2 = \frac{33}{220} Δ U 1 = 0.15 Δ U 1$$

The voltage difference after increasing the capacitance value is only 0.15 times of that before replacing the capacitors, greatly increasing the power supply capacity and ensuring the normal operation of the capsule endoscope throughout the entire digestive tract examination process.

There is also a power switch 14 provided between the power supply component 11 and the power storage component 15, which is used to control the connection and disconnection between the power supply component 11 and the power storage component 15, and the power utilization component as a whole. When the power switch 14 is disconnected, the connection between the power supply component 11 and the power storage component 15, as well as between the power supply component 11 and the power utilization component, are both in a disconnected state. When the capsule needs to be turned on, the power switch 14 is controlled to be closed by an external signal such as infrared light irradiation, and the capsule endoscope is activated. During the operation of the capsule endoscope, the power switch 14 usually remains closed.

Preferably, the power supply component 11 is one or more silver oxide batteries. Compared to lithium batteries, silver oxide batteries have advantages such as stable and reliable performance, and there is no need to consider the problems of the higher cost and the insufficiently stable discharge of lithium batteries. The silver oxide batteries when used in conjunction with the above-mentioned power storage component, are fully capable of providing the peak power supply that meets the requirements of the capsule endoscope.

As shown in FIG. 2, a preferred implementation of the first embodiment of the capsule endoscope of the present invention. In the preferred implementation, a voltage regulator 17 is provided between the power storage component 15 and the image acquisition component. The voltage regulator comprises a step-up circuit and/or a step-down circuit. The step-up circuit can be, for example, a Boost circuit (also known as Boost Chopper or Boost Converter), and the step-down circuit can be, for example, a Buck circuit (also known as Buck Converter). When a large-capacity capacitor is selected for the power storage component 15, the voltage regulator 17 can increase the service life of the capacitor while stabilizing the voltage.

The power utilization component also comprises other power utilization functional components 21. The functional components 21 may be, for example, antenna, image processer, etc., which generally consume relatively little energy during operation.

A second electronic switch 20 is provided between the power supply component 11 and other power utilization functional component 21. The connection between the functional component 21 and the high-power supply components such as the illumination component 12 is mainly in parallel. The functional component 21 is usually powered only by the power supply component 11. The second electronic switch 20 is used to control the connection and disconnection between the power supply component 11 and the functional component 21. When the high-power-consumption power utilization components, such as the illumination component 12 and the camera component 13, experience insufficient power during operation, the second electronic switch 20 is controlled to disconnect by the instruction of the control component of the capsule endoscope, which can cut off the power supply from the power supply component 11 to the functional component 21, thereby further enhancing the energy supply of the energy group of the capsule endoscope to the high-power-consumption components and further increasing peak current.

In addition, the first electronic switch 16 and the second electronic switch 20 may also be integrated with the power utilization components they control, respectively, and working instructions/sleep instructions can be sent to the power utilization components by software configuration, so that the power utilization components can be controlled to be in an operating state or a sleeping state.

As shown in FIG. 3, another preferred implementation of the first embodiment of the capsule endoscope of the present invention. In the preferred implementation, there is a third electronic switch 22 provided between the power supply component 11 and the power storage component 15. The third electronic switch 22 is used to control the connection and disconnection between the power supply component 11 and the power storage component 15, as well as between the power supply component 11 and the illumination component 12 and the camera component 13. The third electronic switch 22 can be used in conjunction with the first electronic switch 16. The function of the third electronic switch 22 is that when the power supply component 11 and the power storage component 15 together form a power pack to supply power to the illumination component 12 and the camera component 13, both the third electronic switch 22 and the first electronic switch 16 are in the closed state. At this time, the power supply component 11 may also need to supply power to other power utilization components besides the illumination component 12 and the camera component 13, and if the power supply component 11 continues to supply a large current to the illumination component 12 and the camera component 13 at the same time, the other power utilization components may not operate properly. Therefore, by disconnecting the third electronic switch 22, only the power storage component 15 supplies power to the illumination component 12 and the camera component 13 at this time, while the power supply component 11 only supplies power to other power utilization components. That is to say, by controlling the third electronic switch 22, it is possible to select different power supply modes. When the third electronic switch 22 is closed, the first electronic switch 16 is closed, and at this time, the power supply component 11 and the power storage component 15 jointly supply power to the illumination component 12 and the camera component 13. When the first electronic switch 16 is closed and the third electronic switch 22 is disconnected, at this time, only the power storage component 15 supplies power to the illumination component 12 and the camera component 13, and the power supply component 11 only needs to supply power to other power utilization components, thereby ensuring that all power utilization components can operate normally.

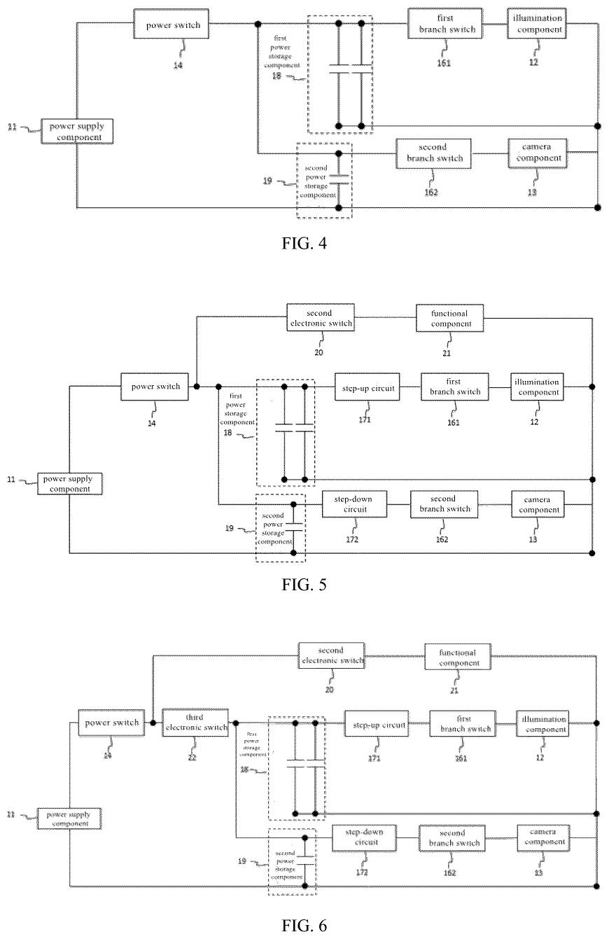

As shown in FIG. 4, a schematic diagram of the principle of the second embodiment of the capsule endoscope of the present invention. In this embodiment, the capsule endoscope comprises a first power storage component 18 and a second power storage component 19, wherein the first power storage component 18 is connected in parallel with the power supply component 11 to form a first power pack, which supplies power to the illumination component 12, and the second power storage component 19 is connected in parallel with the power supply component 11 to form a second power pack, which supplies power to the camera component 13. By setting up dedicated power packs for different power utilization components, the capsule endoscope can achieve on-demand power distribution, thereby improving energy efficiency and safety. For power utilization components with higher power consumption, more capacitors can be configured to supply power to them. When the illumination component 12 is operating, the power supply component 11 supplies power to the illumination component 12, and the electrical energy stored in the capacitor of the first power storage component 18 is released to simultaneously supply power to the illumination component 12, ensuring that the illumination component 12 can receive sufficient power. When the camera component 13 is operating, the power supply component 11 supplies power to the camera component 13, and the electrical energy stored in the capacitor of the second power storage component 19 is released to simultaneously supply power to the camera component 13, ensuring that the camera component 13 also can receive sufficient power.

In this embodiment, a dual capacitor form is used for the first power storage component 18 that supplies power to the illumination component 12, and a single capacitor form is used for the second power storage component 19 that supplies power to the camera component 13. However, it is important to understand that the number of capacitors in the first power pack and the second power pack is determined by the power consumption of the power utilization components they supply power to, as well as the capacitance value of the capacitors themselves, and is not limited to the above situation. In general, the number of capacitors in the power pack corresponding to the high-power-consumption power utilization component is greater than the number of capacitors in the power pack corresponding to the low-power-consumption power utilization component.

The first electronic switch 16 of the capsule endoscope comprises or consists of a first branch switch 161 and a second branch switch 162. The first branch switch 161 is disposed between the first power pack and the illumination component 12, for controlling the connection and disconnection between the first power pack and the illumination component 12. The second branch switch 162 is disposed between the second power pack and the camera component 13, for controlling the connection and disconnection between the second power pack and the camera component 13.

Specifically, when the first branch switch 161 is disconnected, the power supply component 11 charges the first power storage component 18, but does not power the illumination component 12. When the first branch switch 161 is closed, the status of the first power storage component 18 changes, and the power pack composed of the power supply component 11 and the first power storage component 18 together supplies power to the illumination component 12. By setting the first branch switch 161, it is possible to accurately control the provision time of the peak current. Only during the operation of the illumination component 12, the first power storage component 18 can be combined with the power supply component 11 to jointly supply power to the illumination component 12, thereby increasing the energy utilization efficiency under the premise of increasing the peak current, and avoiding electrical energy waste.

Similarly, when the second branch switch 162 is disconnected, the power supply component 11 charges the second power storage component 19, but does not supply power to the camera component 13. When the second branch switch 162 is closed, the status of the second power storage component 19 changes, and the power pack composed of the power supply component 11 and the second power storage component 19 together supplies power to the camera component 13. By setting the second branch switch 162, it is possible to accurately control the provision time of the peak current. Only during the operation of the camera component 13, the second power storage component 19 can be combined with the power supply component 11 to jointly supply power to the camera component 13.

As shown in FIG. 5, a preferred implementation of the second embodiment of the capsule endoscope of the present invention. In the preferred implementation, the step-up circuit 171 is disposed between the first power pack and the illumination component 12, and the step-down circuit 172 is disposed between the second power pack and the camera component 13. The step-up circuit 171 may be, for example, a Boost circuit, including inductors, capacitors, etc. The illumination component has a high current when in use. If the current supply is insufficient, it is prone to automatically shut off, thereby unable to provide lighting. By boosting operation through the step-up circuit 171, in conjunction with the first power storage component 18, it ensures that the illumination component 12 has sufficient current, thereby ensuring the brightness and stability of the lighting. The step-down circuit 172 may be, for example, a Buck circuit, including inductors, diodes, capacitors, etc. In the case of high power consumption, if the voltage of the camera component 13 is unstable, the image quality may deteriorate during long-term capturing. By setting up the step-down circuit 172 in conjunction with the second power storage component 19, the voltage can be stabilized, thereby improving the quality of captured images.

The power utilization component also comprises other power utilization functional components 21. A second electronic switch 20 is provided between the power supply component 11 and the functional component 21, and the second electronic switch is used to control the connection and disconnection between the power supply component and the functional components. The connection relationships and operating principles of the components described in this preferred embodiment have been detailed in the description, and do not be reiterated here.

As shown in FIG. 6, another preferred implementation of the second embodiment of the capsule endoscope of the present invention. In the preferred implementation, a third electronic switch 22 is also provided between the power supply component 11 and the first power storage component 18, and the second power storage component 19. The third electronic switch is used to control the connection and disconnection between the power supply component 11 and the first power storage component 18, and the second power storage component 19, as well as between the power supply component 11 and the illumination component 12 and the camera component 13. The role of the third electronic switch is that when the power supply component 11, the first power storage component 18, and the second power storage component 19 form a power pack to supply power to the illumination component 12 and the camera component 13, the power supply component 11 may also need to supply power to other power utilization components besides the illumination component 12 and the camera component 13. If the power supply component 11 continues to supply a large current to the illumination component 12 and the camera component 13 at the same time, other power utilization components may not operate properly. Therefore, by disconnecting the third electronic switch 22, at this time, only the first power storage component 18 and the second power storage component 19 supply power to the illumination component 12 and the camera component 13 respectively, while the power supply component 11 only supplies power to other power utilization components. In other words, by controlling the third electronic switch 22, it is possible to select different power supply modes. When the third electronic switch 22 is closed, the first branch switch 161 and the second branch switch 162 are also closed. This means that the power supply component 11 and the first power storage component 18 jointly supply power to the illumination component 12, and the power supply component 11 and the second power storage component 19 jointly supply power to the camera component 13. When the first branch switch 161 and the second branch switch 162 are closed, and the third electronic switch 22 is disconnected, at this time, only the first power storage component 18 supplies power to the illumination component 12, the second power storage component 19 supplies power to the camera component 13, and the power supply component 11 only needs to supply power to other power utilization components, ensuring that all power utilization components can operate normally.

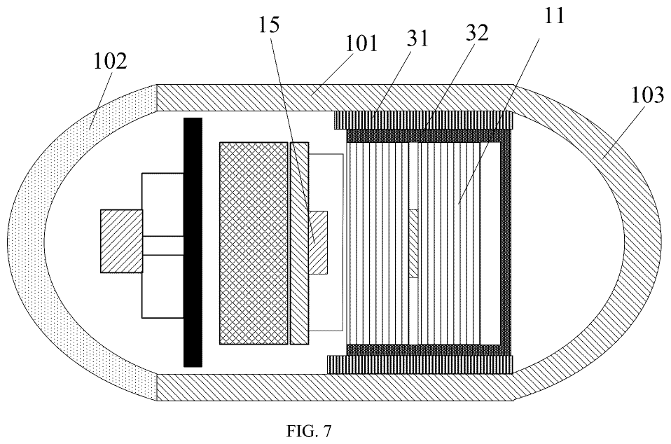

As shown in FIG. 7, a schematic view of the structure of the capsule endoscope according to the embodiments of the present invention. The capsule endoscope comprises an enclosure, the enclosure comprising an enclosure main body portion 101 and enclosure end portions 102, 103, the enclosure end portions 102, 103 are located at both ends of the enclosure main body portion. The enclosure defines an accommodating space to accommodate the power supply component 11 and the power utilization components of the capsule endoscope. A thermal insulation material layer 31 is disposed between the enclosure and the power supply component 11.

For the power supply component (such as silver oxide batteries) in the capsule endoscope, the effect of temperature on the operating time of the capsule is very significant. The main reason is that temperature can accelerate the internal chemical reactions of the silver oxide batteries, ensuring smooth electron migration.

However, since the enclosure of capsule endoscope is primarily made of biocompatible polymer materials, which do not generate heat themselves, the heat generated by the battery operation is transferred to the low-temperature enclosure through air convection, resulting in that the heat from the battery cannot being accumulated, and the temperature cannot to continue to rise. Finally, the equilibrium temperature of the power supply component 11 is lower in the operating state.

To reduce the heat conduction between the power supply component 11 and the enclosure, reduce convection, and decrease the heat radiation of the battery, the thermal insulation material layer 31 is disposed between the enclosure and the power supply component 11.

Setting an thermal insulation material layer 31 between the enclosure and the power supply component 11 may effectively reduce the heat convection between the power supply component 11 and the enclosure, ensuring that the battery may reach a higher thermal equilibrium temperature.

The thermal insulation material layer 31 may be insulating glue, aerogel, etc., with a poor thermal conductivity, or a solid insulating material with a poor thermal conductivity. For example, the thermal insulation material layer may comprise or consist of thermal insulation glue in a gel state, such as nanostructured aerogel, or may comprise or consist of solid glass fibers, mineral wool insulation fibers, calcium silicate, alumina fibers, cellulose thermal insulation element, polystyrene thermal insulation element, and polyurethane foam thermal insulation element.

In this embodiment, the capsule endoscope further comprises a sealing layer 32, the sealing layer 32 is disposed between the thermal insulation material layer 31 and the power supply component 11.

To better protect hardware components such as the power supply component 11 and ensure the effectiveness of the thermal insulation material layer 31, the sealing layer 32 is disposed between the power supply component 11 and the thermal insulation material layer 31. The power supply component 11 is sealed with a sealing layer with a thickness of 0.01 mm-0.02 mm, preventing direct contact between the power supply component 11 and the thermal insulation material layer 31, thereby ensuring the effectiveness of the thermal insulation material layer.

Especially when the thermal insulation material layer is a thermal insulation glue in gel state, if the thermal insulation glue contacts hardware such as the power supply component 11, the thermal insulation glue may not solidify due to the heat generated by the power supply component 11 during operation, affecting the stability of use.

Using a sealing layer 32 to separate the thermal insulation material layer 31 from the power supply component 11 can better maintain the stability of the capsule endoscope. When the thermal insulation material layer 31 is a solid insulating material, the sealing layer 32 can also prevent the influence of dust and other particles on the battery in the thermal insulation material layer, improving the stability of the operation of the power supply component 11.

The material of sealing layer 32 may be polyethylene, polypropylene, polyvinyl chloride, or any other polymer materials. The present invention is not limited to this.

In the present invention, the terms “first” and “second” are only used for descriptive purposes and should not be understood as indicating or implying relative importance; the term “multiple” means two or more. For those skilled in the art, the specific meanings of the terms in the present invention may be understood on a case-by-case basis.

The above description is only some of the preferred embodiments of the present invention and is not intended to limit the present invention. For those of ordinary skill in the art, the present invention may have various changes and variations. Any modifications, equivalent substitutions, improvements, etc. made within the spirit and principles of the present invention should be included in the scope of protection of the present invention.

1. A capsule endoscope, comprising a power supply component and a power utilization component, the power supply component supplying power to the power utilization component, the power utilization component comprising an image acquisition component, further comprising:
at least one power storage component, and a first electronic switch;
the power storage component is connected in parallel with the power supply component to form a power pack, jointly providing power to the image acquisition component, wherein the power storage component is a single capacitor or two or more capacitors connected in parallel; and
the first electronic switch is disposed between the power pack and the image acquisition component, for controlling the connection and disconnection between the power pack and the image acquisition component.

2. The capsule endoscope of claim 1, comprising a first power storage component and a second power storage component, wherein the image acquisition component comprises a camera component and an illumination component, wherein the first power storage component is connected in parallel with the power supply component to form a first power pack, supplying power to the illumination component, and the second power storage component is connected in parallel with the power supply component to form a second power pack, supplying power to the camera component.

3. The capsule endoscope of claim 2, wherein the number of capacitors in the first power pack and the second power pack is determined by the power consumption of the power utilization component they supply power to, and the number of capacitors in the power pack corresponding to the high-power-consumption power utilization component is greater than the number of capacitors in the power pack corresponding to the low-power-consumption power utilization component.

4. The capsule endoscope of claim 2, wherein the first electronic switch comprises a first branch switch and a second branch switch, wherein the first branch switch is disposed between the first power pack and the illumination component, for controlling the connection and disconnection between the first power pack and the illumination component, and the second branch switch is disposed between the second power pack and the camera component, for controlling the connection and disconnection between the second power pack and the camera component.

5. The capsule endoscope of claim 1, further comprising a power switch, wherein the power switch is disposed at the output end of the power supply component, for controlling the connection and disconnection between the power supply component and the power storage component, and the power utilization component; and wherein when the power switch is disconnected, the power supply component and the power storage component, and the power supply component and the power utilization component are both in a disconnected state.

6. The capsule endoscope of claim 1, further comprising a voltage regulator, wherein the voltage regulator is disposed between the power pack and the image acquisition component.

7. The capsule endoscope of claim 6, wherein the voltage regulator comprises a step-up circuit and a step-down circuit, wherein the step-up circuit is disposed between the first power pack and the illumination component, and the step-down circuit is disposed between the second power pack and the camera component.

8. The capsule endoscope of claim 1, further comprising a second electronic switch, wherein the power utilization component further comprises at least one functional component, and the second electronic switch is disposed between the power supply component and the functional component, for controlling the connection and disconnection between the power supply component and the functional component.

9. The capsule endoscope of claim 8, further comprising a third electronic switch, wherein the third electronic switch is disposed between the power supply component and the power storage component, for controlling the connection and disconnection between the power supply component and the power storage component, and between the power supply component and the image acquisition component.

10. The capsule endoscope of claim 1, further comprising a thermal insulation material layer, wherein the thermal insulation material layer is disposed between the power supply component and an enclosure of the capsule endoscope.

11. The capsule endoscope of claim 10, wherein the thermal insulation material layer comprises insulating glue, aerogel material, or solid insulating material.

12. The capsule endoscope of claim 10, further comprising a sealing layer, wherein the sealing layer is disposed between the power supply component and the thermal insulation material layer.
